(19) United States
(12) Patent Application Publication
(10) Pub. No.: US 2002/0127557 A1
(43) Pub. Date: Sep. 12, 2002

(54) Method for identification of cDNAs encoding signal peptides

(76) Inventors: Ruoying Tan, Foster City, CA (US); Glenn Fu, Dublin, CA (US); Michael Rose, Mountain View, CA (US); Juan Zhang, Cupertino, CA (US)

Correspondence Address: Karl Bozicevic, BOZICEVIC, FIELD & FRANCIS LLP, Suite 200, 200 Middlefield Road, Menlo Park, CA, 94025, US

(21) Appl. No.: 09/803,317

(22) Filed: Mar. 9, 2001

(51) Int. Cl. C12Q 1/68; G01N 33/53; C12Q 1/02; C12N 15/00

(52) U.S. Cl. 435/6; 435/7.1; 435/320.1; 435/29

(57) ABSTRACT

The present invention provides a method in which cDNAs that encode signal sequences for secreted or membrane-associated proteins are isolated using a fusion protein that directs secretion of a molecule that provides antibiotic resistance, e.g., β-lactamase. The present method allows the isolation of signal peptide-associated proteins that may be difficult to isolate with other techniques. Moreover, the present method is amenable to throughput screening techniques and automation, and especially in validating the presence of the signal sequence via expression of the protein in both prokaryotic and eukaryotic cells. This invention provides a powerful and approach to the large scale isolation of novel secreted proteins.

### FIELD OF THE INVENTION

The present invention is directed to the field of protein identification and isolation, and more particularly to the identification and isolation of secreted proteins.

### BACKGROUND OF THE INVENTION

Membrane-based and secreted proteins are essential in the formation, differentiation and maintenance of multicellular organisms. Cellular proliferation, migration, differentiation, and interaction are governed by information received from the cells neighbors and the immediate environment. This information is often transmitted by secreted polypeptides (e.g., mitogenic factors, survival factors, cytotoxic factors, differentiation factors, neuropeptides, and hormones) which are in turn received and interpreted by diverse cell receptors. These secreted polypeptides, signaling molecules and cellular receptors must translocate through the plasma membrane to reach their site of action in the extracellular environment.

The targeting of both secreted and transmembrane proteins to the secretory pathway is accomplished via the attachment of a short, amino-terminal sequence, known as the signal peptide, signal sequence or secretory leader sequence. von Heijne, G. (1985) J. Mol. Biol. 184, 99-105; Kaiser, C. A. & Botstein, D. (1986), Mol. Cell. Biol. 6, 2382-2391. The signal peptide itself contains several elements necessary for optimal function, the most important of which is a hydrophobic component. Immediately preceding the hydrophobic sequence is often one or more basic amino acids. The carboxyl-terminal end of the signal peptide has a pair of small, uncharged amino acids separated by a single intervening amino acid which defines the signal peptidase cleavage site.

Secreted and membrane-bound cellular proteins have wide applicability in various industrial applications, including pharmaceuticals, diagnostics, biosensors and bioreactors. Approximately 90% of all drug targets at present are secreted proteins or transmembrane proteins. In addition, most protein drugs commercially available at present, such as thrombolytic agents, interferons, interleukins, erythropoietins, colony stimulating factors, and various other cytokines are secretory proteins. Their receptors, which are membrane proteins, also have potential as therapeutic or diagnostic agents. Significant resources are presently being expended by both industry and academia to identify new native secreted proteins.

While the hydrophobic component, basic amino acid and peptidase cleavage site can usually be identified in the signal peptide of known secreted proteins, the high level of degeneracy within any one of these elements makes it difficult to identify or isolate secreted or transmembrane proteins solely by searching for signal peptides in DNA databases (e.g., GeneBank, GenPept), or based upon hybridization with DNA probes designed to recognize cDNA's encoding signal peptides. A number of different methods have thus been developed to aid in the identification of such proteins.

For example, in Klein R. D. et al. (1996), Proc. Natl. Acad. Sci. 93, 7108-7113 and Jacobs (U.S. Pat. No. 5,536,637 issued Jul. 16, 1996), cDNAs encoding novel secreted and membrane-bound mammalian proteins are identified by detecting their secretory leader sequences using the yeast invertase gene as a reporter system. A mammalian cDNA library is ligated to a DNA encoding a non-secreted yeast invertase, the ligated DNA is isolated and transformed into yeast cells that do not contain an invertase gene. Recombinants containing the non-secreted yeast invertase gene ligated to a mammalian signal sequence are identified based upon their ability to grow on a medium containing only sucrose or only raffinose as the carbon source. The mammalian signal sequences identified are then used to screen a second, full-length cDNA library to isolate the full-length clones encoding the corresponding secreted proteins. While effective, the invertase yeast selection process described above has several disadvantages. First, it requires the use of special SUC2-yeast cells, e.g., in which the SUC2 gene encoding the invertase protein has been deleted or the coding sequence of the native invertase signal has been mutated so that the invertase is not secreted. Second, even invertase-deficient yeast may grow on sucrose or raffinose, albeit at a low rate, therefore, the invertase selection may need to be repeated several times to improve the selection for transformants containing the signal-less yeast invertase gene ligated to a mammalian secretory leader sequence. Third, the invertase selection process is further inadequate because a certain threshold level of enzyme activity needs to be secreted to allow growth. Although 0.6-1% of wild-type invertase secretion is sufficient for growth, certain mammalian signal sequences are not capable of functioning to yield even this relatively moderate level of secretion. Kaiser, C. A. et al. (1987), Science 235; 312-317.

In another example, U.S. Pat. No. 6,136,569 describes a novel method for identifying genes encoding secreted and membrane-bound proteins using a starch degrading enzyme as a reporter molecule. Mammalian signal sequences are detected based upon their ability to effect the secretion of a starch degrading enzyme (e.g., amylase) lacking a functional native signal sequence. The secretion of the enzyme is monitored by the ability of the transformed yeast cells to degrade and assimilate soluble starch. This method, however, also suffers from limitations similar to that of the Klein and Jacobs methods, such as a dependency on secretion levels and function of mammalian signal sequences.

Methods that permit the identification of cDNAs encoding a signal sequence capable of directing the secretion of a particular protein from certain cell eukaryotic types have also been investigated. Honjo, U.S. Pat. No. 5,525,486 describes identification of genes having signal sequences by selecting for secretion of proliferation and/or differentiation factors. McCarthy, et al. U.S. Pat. No. 5,952,171, describe methods of identifying alkaline phosphatase secretion in cells a method for identifying a cDNA nucleic acid encoding a mammalian protein having a signal sequence. The method is a multi-step process, which entails ligating the library of mammalian cDNA to DNA encoding alkaline phosphatase lacking both a signal sequence and a membrane anchor sequence, and transforming bacterial cells with the ligated DNA to create a bacterial cell clone library. The DNA is then isolated from a bacterial cell clone library, and used to separately transfect mammalian cells which do not express alkaline phosphatase to create a mammalian cell clone library so that each clone in the mammalian cell clone library corresponds to a clone in the bacterial cell clone library. Clones in this mammalian cell clone library which express alkaline phosphatase can then be selected for by the presence of alkaline phosphatase in the mammalian cells. These methods are time consuming, however, since they require multiple steps, and the use of mammalian cells as the primary selection mechanism is labor intensive.

Given the great efforts presently being expended to discover novel secreted and transmembrane proteins as potential therapeutic agents, there is a great need for an improved system which can simply and efficiently identify the coding sequences of such proteins in mammalian recombinant DNA libraries. The present invention addresses this need.

### SUMMARY OF THE INVENTION

The present invention provides a method in which cDNAs that encode secreted and/or membrane-associated proteins are isolated using a vector comprising a leaderless protein that confers antibiotic resistance when secreted from the host cell. Insertion of a cDNA encoding a signal sequence directs secretion of the fusion protein to confer antibiotic resistance, e.g. by secretion of β-lactamase. The present method allows the isolation of signal peptide-associated proteins that may be difficult to isolate with other techniques. The present method is amenable to throughput screening techniques and automation, and especially in validating the presence of the signal sequence via expression of the protein in both prokaryotic and eukaryotic cells. This invention provides a powerful approach to the large scale isolation of novel secreted and/or transmembrane proteins.

In a first embodiment, the invention provides a method of identifying a cDNA encoding a secreted or transmembrane protein using a microbial selection system. The methods comprise 1) formation of a fusion nucleic acid comprising a cDNA and a nucleic acid encoding a leaderless selection protein in a prokaryotic host, 2) production of the fusion protein encoded by the fusion nucleic acid in the host; 3) secretion of a fusion protein of a cDNA comprising a signal sequence and a selection marker, and 4) subsequent selection of host growth based on secretion of the fusion protein.

In a specific embodiment, the invention features a method for isolating cDNAs that encode secreted or transmembrane mammalian proteins in a bacterial host. A cDNA nucleic acid encoding a protein that potentially encodes a signal sequence is directionally introduced into a vector which comprises a nucleic acid encoding a leaderless secretable selection protein (e.g., leaderless β-lactamase). Bacterial cells are transformed with the vector having the inserted cDNA, and cultured in a selection medium (e.g., medium containing a β-lactam antibiotic such as ampicillin) and determining growth of the bacterial cells in said selection medium. A signal sequence in the cDNA will allow secretion of the fusion protein produced by the vector (e.g., the β-lactamase fusion protein), which in turn will allow growth of the bacterial cells in the selection medium. Growth of the bacteria in the selection medium is thus indicative of a signal sequence in said cDNA. Following selection, the vector is generally isolated from the bacterial cell for determination of the cDNA sequence and other molecular analysis. The cDNA insert can be directly analyzed in the vector, or it may be further isolated from the vector sequences (e.g., by PCR) for investigation.

In a particular embodiment, the invention features a method for constructing bacterial library enriched with cDNAs that encode a protein having a signal sequence. The method comprises 1) production of cDNAs; 2) directionally introducing each of the produced cDNAs into a vector that comprises a nucleic acid insert encoding a leaderless secretable selection protein to produce a cDNA-leaderless secretable selection protein fusion; 3) transforming bacterial cells with the vectors containing the cDNA inserts; 4) allowing expression of the fusion nucleic acid in the bacterial cells; and 5) selecting bacterial cells containing a cDNA encoding a signal sequence by growth in a selection medium. The cDNAs used may be produced using any number of methods known in the art, but are preferably methods that produce 5′-biased cDNAs. Bacteria transformed with a vector having a cDNA encoding a signal sequence can be identified by their ability to grow in a medium containing a growth selection compound, e.g., an antibiotic.

In another particular embodiment, the methods of the invention can be used to specifically identify and/or isolate cDNAs encoding transmembrane proteins. A fusion protein having an intracellular selection protein will not necessarily allow for proper selection for a signal peptide, as the selection protein must be extracellularly located to have the appropriate selection activity. cDNAs encoding transmembrane proteins can thus be identified using a method comprising 1) introducing a 5′ fragment of the cDNA into a selection vector and selecting for secretion of a fusion protein using the methods of the present invention and 2) introducing a complete cDNA or a 5′ portion of a cDNA thought to encode a transmembrane region into a selection vector, and selecting for secretion of a fusion protein using the methods of the present invention. cDNAs encoding both a signal sequence and a transmembrane domain will be selected for in the first instance, but will not allow growth in the selection medium when the selectio protein is fused to the intracellular region as in the second instance.

The present invention also provides a dual expression vector comprising a leaderless secretable selection protein, e.g. a dual expression vector having an insert encoding leaderless β-lactamase. Such a vector can be used to validate the secretion of a protein, having a known or unknown function, by creating a fusion protein that can be used to identify secretion of the protein encoded by a cDNA. The cDNA sequence can be directionally cloned into the vector to produce a nucleic acid fusion having the cDNA at the 5′ end and a leaderless secretable selection protein in frame 3′ to the cDNA. In addition, this vector can be used for large-scale identification of cDNAs encoding proteins having signal sequences, e.g., for the production of a cDNA library to identify secreted proteins in a particular cell or tissue type. The vector may also be used to indicate the ability of a protein to remain in a plasma membrane, i.e. to not be secreted, as a fusion protein having a transmembrane region 5′ of the selection protein will not allow translocation of this portion of the protein across the plasma membrane.

In a preferred embodiment, the methods of the present invention utilize a dual expression vector which allows expression in both prokaryotic and eukaryotic systems. Following identification of cDNAs encoding protein with signal sequences using bacterial selection, the secretion can be directly confirmed via tranfection of the vector into a eukaryotic system (e.g., a mammalian system) and detection of the secreted fusion protein. Expression of the secreted protein in a eukaryotic system can be determined using an assay (e.g., a hydrolysis assay), or via a direct assay of the protein in supernatants from the transfected cells for presence of secreted selection protein. Following this selection, the vector encoding the cDNA fusion can be isolated and sequenced or otherwise analyzed.

An object of the present invention is to identify cDNAs encoding secreted and/or transmembrane proteins.

Another object of the invention is to provide a cDNA library which is highly enriched in cDNAs encoding signal sequences.

Yet another object of the invention is to provide a vector useful in the validation of protein secretion in bacterial and mammalian expansion systems and for the production of cDNA libraries enriched in nucleic acids encoding secreted and transmembrane proteins.

An advantage of the invention is that it provides fast and effective methods for selecting cDNAs encoding proteins having signal sequences, and for identifying new secreted proteins.

Yet another advantage of the present invention is that the selection process using a leaderless secretable selection protein is fast and cost-effective.

Another advantage of the invention is that dual expression vectors allow for direct confirmation of a signal sequence in both a prokaryotic (e.g., bacterial) and eukaryotic (e.g., mammalian) system using a single construct.

These and other objects, advantages, and features of the invention will become apparent to those persons skilled in the art upon reading the details of the invention as more fully described below.

### BRIEF DESCRIPTION OF THE DRAWINGS

FIG. 1 is a schematic drawing of a portion of a pBK-CMV-leaderless β-lactamase vector having a directionally cloned cDNA insert.

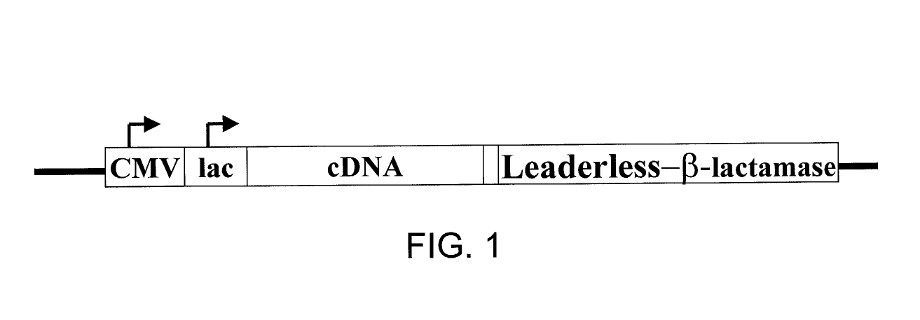

FIG. 2 is series of schematic drawings of the pBK-CMV-leaderless β-lactamase constructs comprising various inserts illustrating the ability of these constructs to grow in a selection medium and the ability to secrete β-lactamase in prokaryotic and eukaryotic cells.

### DETAILED DESCRIPTION OF PREFERRED EMBODIMENTS

Before the present methods, constructs and system are described, it is to be understood that this invention is not limited to particular methods, constructs and system described, and as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “bacteria” includes a single bacterium as well as a plurality of such bacteria, and reference to “the selection protein” includes reference to one or more different proteins and equivalents thereof known to those skilled in the art, and so forth.

### Definitions

The terms “polynucleotide” and “nucleic acid”, used interchangeably herein, refer to a polymeric forms of nucleotides of any length, either ribonucleotides or deoxynucleotides. Thus, these terms include, but are not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. These terms further include, but are not limited to, mRNA or cDNA that comprise intronic sequences (see, e.g., Niwa et al. (1999) Cell 99(7):691-702). The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidites and thus can be an oligodeoxynucleoside phosphoramidate or a mixed phosphoramidate-phosphodiester oligomer. Peyrottes et al. (1996) Nucl. Acids Res. 24:1841-1848; Chaturvedi et al. (1996) Nucl. Acids Res. 24:2318-2323. A polynucleotide may comprise modified nucleotides, such as methylated nucleotides and nucleotide analogs, uracyl, other sugars, and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones.

The term “selection medium” as used herein refers to a growth medium for a cell that contains a substance that is generally restricts or inhibits growth of a host cell. For example, a selection medium may contain an antibiotic, such as ampicillin.

The term “selection protein” as used herein refers to a protein that, upon expression in a cell, confers an ability to survive in a selection medium, i.e. in an environment in which the cell cannot survive without production of the selection protein in an effective manner, e.g., without secretion of the selection protein. An example of such a selection protein is β-lactamase, which upon secretion allows a cell to survive in a medium containing a β-lactam antibiotic such as ampicillin. Selection proteins for use with the present invention can be known selection proteins or identified by various known methods, e.g., by detecting differences in growth (e.g, as measured by growth rate) between host cells that differ in target gene product dosage. See, e.g., U.S. Pat. No. 6,046,002.

The term “leaderless secretable selection protein” as used herein refers to a selection protein which has been altered to lack a signal sequence at the N-terminus, and thus has lost the ability to be secreted from a cell upon production. For example, a leaderless secretable selection protein can be a protein that normally confers antibiotic resistance to a bacteria upon secretion (e.g., β-lactamase), but which is lacking the N-terminal signal sequence that allows insertion of the protein through the plasma membrane.

By “directionally cloning”, “directionally cloned” and the like as used herein is meant that a cDNA molecule is inserted 5′ of a nucleic acid encoding a selection protein in a vector such that the nucleic acid encoding the selection protein is in-frame with the cDNA insert for translation purposes. Directional cloning of the cDNA using the methods herein results in a nucleic acid insert encoding a fusion protein of the protein encoded by the cDNA at the N-terminus and a leaderless selection protein at the carboxy terminus. For example, a cDNA can be produced with restriction sites that allow the cDNA to be inserted in a 5′ to 3′ coding orientation using the restriction sites in a vector, e.g., into a multiple cloning site in a vector.

A “host cell”, as used herein, refers to a microorganism or a eukaryotic cell or cell line cultured as a unicellular entity which can be, or has been, used as a recipient for a recombinant vector or other transfer polynucleotides, and include the progeny of the original cell which has been transfected. It is understood that the progeny of a single cell may not necessarily be completely identical in morphology or in genomic or total DNA complement as the original parent, due to natural, accidental, or deliberate mutation.

### General Aspects of the Invention

The method of the invention relies upon the observation that the majority of secreted and membrane-associated proteins possess at their amino termini a stretch of hydrophobic amino acid residues referred to as the “signal sequence.” The signal sequence directs secreted and membrane-associated proteins to a sub-cellular membrane compartment termed the endoplasmic reticulum, from which these proteins are dispatched for secretion or presentation on the cell surface.

A distinct advantage of the invention is that the vector system used allows initial selection of cDNAs in a microbial system and verification in a eukaryotic system. This provides a quick and inexpensive screen for secreted and transmembrane proteins without having to screen a large number of cDNAs potentially encoding a secreted and/or transmembrane protein in a eukaryotic system.

The methods of the invention entail multiple steps, each of which may have a number of variations. These steps are described below in more detail.

Preparation of cDNA

A number of methods for preparing and/or isolating cDNA can be used, as will be apparent to one skilled in the art upon reading the present disclosure.

In general, preparing the first strand cDNA, a primer is contacted with the mRNA with a reverse transcriptase and other reagents necessary for primer extension under conditions sufficient for first strand EDNA synthesis to occur.

Although both random and specific primers (e.g., an oligo dT primer that provides for hybridization to a polyA tail of an mRNA) may be employed, the primer will be sufficiently long to provide for efficient hybridization to the mRNA to first strand synthesis. Where the primers used are random primers, the length of the primers are generally shorter than specific primers, e.g., random hexamers. Specific primers may vary where the primer will typically range in length from 10 to 25 nt in length, usually 10 to 20 nt in length, and more usually from 12 to 18 nt length. Additional reagents that may be present include: dNTPs; buffering agents, e.g. Tris-Cl; cationic sources, both monovalent and divalent, e.g. KCl, MgCl₂; sulfhydril reagents, e.g. dithiothreitol; and the like. A variety of enzymes, usually DNA polymerases, possessing reverse transcriptase activity can be used for the first strand cDNA synthesis step. Examples of suitable DNA polymerases include the DNA polymerases derived from organisms selected from the group consisting of a thermophilic bacteria and archaebacteria, retroviruses, yeasts, Neurosporas, Drosophilas, primates and rodents. Preferably, the DNA polymerase will be selected from Moloney murine leukemia virus (Mo-MLV) as described in U.S. Pat. No. 4,943,531 and Mo-MLV reverse transcriptase lacking RNaseH activity as described in U.S. Pat. No. 5,405,776 (the disclosures of which patents are herein incorporated by reference), human T-cell leukemia virus type I (HTLV-I ), bovine leukemia virus (BLV ), Rous sarcoma virus (RSV ), human immunodeficiency virus (HIV) and Thermus aquaticus (Taq) or Thermus thermophilus (Tth) as described in U.S. Pat. No. 5,322,770, the disclosure of which is herein incorporated by reference, avian reverse transcriptase, and the like. Suitable DNA polymerases possessing reverse transcriptase activity may be isolated from an organism, obtained commercially or obtained from cells which express high levels of cloned genes encoding the polymerases by methods known to those of skill in the art, where the particular manner of obtaining the polymerase will be chosen based primarily on factors such as convenience, cost, availability and the like. Of particular interest because of their commercial availability and well characterized properties are avian reverse transcriptase and Mo-MLV.

In a preferred embodiment, the methods of the present invention utilize 5′ biased cDNAs or full-length cDNAs, as these will be highly enriched in cDNAs containing signal sequences. Exemplary strategies for producing 5′-biased and/or full length cDNAs are described in: copending application U.S. Ser. No. 09/352,540; Edery, et al., “An efficient strategy to isolate full-length cDNAs based on an mRNA cap retention procedure (CAPture),” Mol Cell Biol (June, 1995)15(6):3363-71; Suzuki et al., “Construction and characterization of a full length-enriched and a 5′-end-enriched cDNA library,” Gene (Oct. 24, 1997) 200(1-2):149-56; Alphey, “PCR-based method for isolation of full-length clones and splice variants from cDNA libraries,” Biotechniques (March 1997)22(3):481-4, 486; Carninci et al., “High efficiency selection of full-length cDNA by improved biotinylated cap trapper,” DNA Res (Feb. 28, 1997) 4(1):61-6; Carninci et al., “High-efficiency full-length cDNA cloning by biotinylated CAP trapper,” Genomics (Nov. 1, 1996)37(3):327-36; Schmid et al., “A procedure for selective full length cDNA cloning of specific RNA species,” Nucleic Acids Res (May 26, 1987)15(10):3987-96; Seki et al., “High-efficiency cloning of Arabidopsis full-length cDNA by biotinylated CAP trapper,” Plant J (September 1998) 15(5):707-20; Okayama et al., “High-efficiency cloning of full-length cDNA,” Mol Cell Biol (February 1982) 2(2):161-70; Sekine et al., “Synthesis of full-length cDNA using DNA-capped mRNA,” Nucleic Acids Symp Ser (1993) (29):143-4. Other methods for production of 5′-biased cDNAs can also be used, as will be apparent to one skilled in the art upon reading the present disclosure.

The order in which the reagents are combined may be modified as desired. One protocol that may be used involves the combination of all reagents except for the reverse transcriptase on ice, then adding the reverse transcriptase and mixing at around 4° C. Following mixing, the temperature of the reaction mixture is raised to 37° C. followed by incubation for a period of time sufficient for first strand cDNA primer extension product to form, usually about 1 hour.

Following first strand cDNA synthesis, the resultant duplex mRNA/cDNA (i.e. hybrid) is then contacted with an RNAse capable of degrading single stranded RNA but not RNA complexed to DNA under conditions sufficient for any single stranded RNA to be degraded. A variety of different RNAses may be employed, where known suitable RNAses include: RNAse T1 from Aspergillus orzyae, RNase I, RNase A and the like. The exact conditions and duration of incubation during this step will vary depending on the specific nuclease employed. However, the temperature is generally between about 20 to 37° C., and usually between about 25 to 37° C. Incubation usually lasts for a period of time ranging from about 10 to 60 min, usually from about 15 to 60 min.

Nuclease treatment results in the production of blunt-ended mRNA/cDNA duplexes or hybrids. In the resultant mixture, those mRNA/cDNA hybrids that include a full length cDNA will have the 5′ cap structure of the template mRNA, while those in which a full length cDNA was not produced in the reverse transcription step will not. Following production of the blunt-ended mRNA/cDNA hybrids, the resultant hybrids are then contacted with the fusion protein and isolated as described above.

Following isolation, the nucleic acids may be further processed as desired, where further processing includes: release from the solid phase support (if present), e.g. by cleavage reaction, disruption of the specific bond, and the like; production of double stranded cDNA, etc., where protocols for performing such operations are well known to those of skill in the art.

In a particular embodiment, the cDNA used in the methods of the invention is mammalian cDNA. The mRNA can be isolated from any desired tissue or cell type. For example, peripheral blood cells, primary cells, tumor cells, or other cells may be used as a source of mRNA.

Although the present invention is described throughout in terms of using a cDNA insert, it is also well within the skill of one in the art to use a genomic DNA region as the insert in a vector containing a leaderless secretable selection protein. This may be performed, for example, to enhance expression should a promoter element be present within an intronic region of a gene. The genomic region must fuse with the nucleic encoding the secretable selection protein in a manner that allows expression of a fusion protein incorporating the leaderless selection protein.

Directional Cloning of cDNAs in an Expression Vector

A cDNA is operably inserted into an expression vector to allow detection of an encoded signal sequence by inserting the cDNA is 5′ of the leaderless selection protein in a coding orientation, i.e., is “directionally inserted”. The expression vector encoding the leaderless selection protein can be any vector with a “prokaryotic promoter”, i.e. the ability to express in a desired prokaryotic host, e.g., bacteria or phage. In a particular embodiment, the expression vector is a dual expression vector, i.e. it has the ability to express the inserted cDNA in both a prokaryotic and a eukaryotic system. Preferably, the prokaryotic expression system allows expression in bacteria to allow for antibiotic resistance selection. The eukaryotic expression system comprises a “eukaryotic promoter” which allows for expression in a eukaryotic cell. A eukaryotic promoter need not be a promoter from a eukaryote per se, it just must confer the ability to express a protein in a eukaryotic cell (e.g., a promoter of viral origin such as CMV). The eukaryotic promoter may be adapted for expression in eukaryotic cells such as insect cells or, preferably, mammalian cells. Such mammalian cells can be any suitable mammalian cells, e.g., CHO cells, COS cells, mouse L cells, Hela cells, VERO cells, mouse 3T3 cells, and 293 cells.

Exemplary dual expression vectors that can be used with the present invention include, but are not limited to, STRATAGENE vectors pBK-CMV, in which prokaryotic expression is driven by the lac promoter and mammalian expression is driven by the CMV promoter; pBK-RSV, in which prokaryotic expression is driven by the lac promoter and mammalian expression is driven by the RSV-LTR promoter; and pDual™ Expression System, in which prokaryotic expression is driven by a hybrid T7/lacO promoter and mammalian expression is driven by the CMV promoter.

The expression vectors of the invention comprise a nucleic acid encoding a leaderless secretable selection protein that, upon translation from the vector, produces a defective selection protein that cannot be secreted. In a specific embodiment, the leaderless secretable selection protein is a leaderless β-lactamase that is missing the first 23 amino acids from the wild-type sequence. A nucleic acid encoding this leaderless β-lactamase in inserted into a vector using molecular techniques well known in the art. A cDNA is the inserted into the expression vector such that it is 5′ of the nucleic acid encoding the selection protein and in-frame with the selection protein for translation. Thus, upon translation of the vector coding sequences will produce a fusion protein having the protein encoded by the cDNA at the N-terminus and the selection protein at the carboxy-terminus. Upon secretion of the protein encoded by the cDNA, the selection protein is also secreted to the extracellular region where it has its selection activity.

The expression vector of the invention can also comprise additional elements—linker/multiple cloning sites, additional selectable markers, and origin of replication.

In a particular embodiment, the cDNA insert is a partial cDNA comprising the 5′-most sequences of the cDNA. Upon insertion of this partial cDNA, the vector allows expression of a protein having a signal sequence, but having a truncation such that no transmembrane region that is potentially in the protein is produced. Insertion of a full-length cDNA, or a cDNA fragment that potentially encodes a transmembrane region, can verify the presence of a transmembrane region encoding region in a cDNA, as the full-length protein will not allow secretion of the selection protein to the extracellular region.

Selection Proteins

In general, the selection protein can be any protein that, upon secretion, provides for positive selection of the host cell in a selection medium.

Drug inactivation is an important mechanism of resistance against β-lactam antimicrobials, aminoglycosides, and chloramphenicol and it generally involves the hyperproduction and secretion of an enzyme (i.e. a “selectable protein”) that inactivates the drug. Bacteria can resist antimicrobial chemicals by mechanisms such as: inactivating the drugs with secreted selection proteins; reducing drug access sites of action by virtue of membrane characteristics; altering the drug target so that the antimicrobial no longer binds to it; and bypassing the drug's metabolism.

For example, bacteria can resist antimicrobial chemicals, and thus acquire antibiotic resistance, by secreting proteins such as β-lactamases, acetylases, adenylases, and phosphorylases. Any such secreted proteins that provide for antimicrobial resistance are suitable for use in the invention as a selection protein following modification of the coding sequences to remove the leader sequence. The sequences for exemplary secreted antibiotic resistance genes are available and methods for the removal of the signal sequence are well known in the art, and one skilled in the art will be able to use such upon reading the present disclosure.

In specific embodiments, the selection protein used is a β-lactamase. β-lactamases are almost ubiquitous in bacteria and are found in both gram-positive and gram-negative microbes. The β-lactam antimicrobials (penicillins, cephalosporins, carbapenems, monobactams) all bind to transpeptidase and inhibit peptidoglycan and thus cell-wall synthesis. There are many β-lactamases, with the most important classes being 1 and 4. (Richmond M H and Sykes R B., The beta-lactamases of gram-negative bacteria and their possible physiological role, Adv Microb Physiol.; 9:31-88 (1973); Bush K. Characterization of beta-lactamases, Antimicrob Agents Chemother:33:259-76 (1989). In a particular embodiment, the enzyme is inducible, i.e. secretion of the enzyme occurs only in the presence of an inducer or constitutive output. The β-lactam antimicrobials used in selecting the will depend in part upon the β-lactamase gene chosen for use in the vector.

Following vector preparation, the vector is introduced into the host. Introduction of the vector into the host may be accomplished using any convenient methodology. For example, electroporation as described in Dower et al., Nuc. Acids Res. (1988) 16:6127 is one preferred method of introducing vector DNA into the host cell. Other techniques of interest that may find use include those described in: Cohen et al., Proc. Nat'l. Acad. Sci. USA (1972) 69:2110; Hanahan, J. Mol. Biol. (1983) 166:557-580; Graham and Van der Eb, Virology (1973) 52:456; Wang et al., Science (1985)228:149; Sompayrac et al., Proc. Nat'l Acad. Sci. USA (1981) 78:7575-7578 and Felgner et al., Proc. Nat'l Acad. Sci. USA (1987)84:7413.

Where the vector employed is a phagemid, the subject methods will further comprise co-introducing helper phage into the host, where the helper phage will carry the full complement of the capsid encoding genes of the virion to be produced but will be defective in replication. Helper phage that find use will necessarily depend on the nature of the phagemid and the virion to be produced. For example, where the virion to be produced is a filamentous phage, helper phage that find use include M13 helper phage, such as M13KO7, VCS, 1PHer S, and the like.

The resultant transformed hosts will then be allowed to produce the fusion product encoded by the expression system. Following introduction of the vector into the host, the host cells are exposed to a selection medium, either a solid medium (e.g., an agar plate) or a liquid and grown under conditions sufficient for transcription and translation of the genetic information comprised in the vector. For example, bacterial cells producing a secreted β-lactamase fusion protein can be selected by plating the transformed bacteria on an agar plate containing ampicillin and incubating the cells overnight at 37° C. Suitable conditions for the growth and selection of host cells will be apparent to those skilled in the art upon reading this disclosure.

Expression of the Resulting cDNA Library Mammalian Cells for Validation.

The vectors in selected prokaryotic clones can be introduced into eukaryotic cells for validation of the secretion of the selection protein. The vectors can be introduced into suitable host cells using a variety of techniques which are available in the art, such as transferrin polycation-mediated DNA transfer, transfection with naked or encapsulated nucleic acids, liposome-mediated DNA transfer, intracellular transportation of DNA-coated latex beads, protoplast fusion, viral infection, electroporation, gene gun, calcium phosphate-mediated transfection, and the like.

The method for detection of the secreted selection protein will be dependent upon the activity of the selection protein. For example, β-lactamase can be detected in media culture by its ability to hydrolyze the amide bond in the beta-lactam ring of the compound nitrofectin. This hydrolysis causes the medium to undergo a distinctive color change from yellow to red. In addition, the selection protein can be directly detected from media aspirated from the transfected cells.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Vector Construction and Selection of cDNAs Encoding Secreted Fusion Proteins

A vector expressing β-lactamase under a CMV promoter was generated using the vector pBK-CMV (STRATAGENE). The ATG at position 1183 was removed from pBK-CMV vector to prevent non-selected translation, and an EcoRI site was created in the 3′ end of lac promoter. A PCR-generated β-lactamase nucleic acid was engineered to have an EcoRI site at the 5′ end and a KpnI site at the 3′ end. This fragment was inserted between the EcoRI and KpnI sites of the modified pBK-CMV parent vector described above. The modified vector expressing a leaderless-β-lactamase gene, in which the N-terminal 23 amino acids signal sequences were deleted was generated with an EcoRI site at the 5′ end and a KpnI site at the 3′ end. This engineered leaderless β-lactamase fragment was inserted the between EcoRI and KpnI sites of the modified pBK-CMV vector. This vector was called pBK-CMV-leaderless-β-lactamase. This modified parent vector was then used in the production of different vector constructs having inserts, as described below.

1. pBK-CMV-Signal-β-Lactamase

As a first positive control, the gene fragment encoding the β-lactamase signal peptide was synthesized, the fragment having an engineered EcoRI site at the 5′ end and a NotI site at the 3′ end. This fragment was inserted into the parent vector via the EcoRI and KpnI sites to produce a vector having a β-lactamase with a proper signal sequence to provide secretion of β-lactamase.

2. pBK-CMV-CD4-β-Lactamase

As a second positive control, a leaderless β-lactamase fused to a gene known to have a leader sequence, CD4, was synthesized. PCR was used to generate a nucleic acid encoding the CD4 gene, and an EcoRI site was engineered into the 5′ end of the nucleic acid and a NotI site was engineered into the 3′ end. PCR was then used to generate another leaderless-β-lactamase gene, in which the first 23 amino acids (signal leader) were deleted, and a NotI site was engineered at the 5′ end of the β-lactamase coding sequence and a KpnI site was engineered at the 3′ end. The generated CD4 nucleic acid and the leaderless-β-lactamase nucleic acid were connected in NotI site to become CD4-leaderless-β-lactamse fusion gene. This fusion gene was inserted the leaderless-β-lactamase fusion gene between EcoRI and KpnI sites of the modified pBK-CMV vector.

3. pBK-CMV-HSP-β-Lactamase.

PCR was used to generate a nucleic acid corresponding to the coding region of the HSP gene, the PCR product having an EcoRI site at the 5′ end and a NotI site at the 3′ end. EcoRI and NotI were used to excise CD4 gene from the pBK-CMV-CD4-leaderless-β-lactamase construct. The HSP nucleic acid was inserted between the EcoRI and NotI sites of the pBK-CD4-β-lactamase vector.

Prokaryotic Culture and β-Lactamase Activity Assay.

The generated vectors were then transformed into E. Coli using conventional methods. Transformed E. coli was grown on LB-agar plates containing 30 μg/ml kanamycin (non-selected) or 100 μg/ml carbenicillin and 1 mM IPTG (selected). Colonies were picked and grown in the frizzing medium+supplement (Incyte medium kitchen) containing 30 μg/ml kanamycin or in the frizzing medium+supplement containing 100 μg/ml carbenicillin and 1 mM IPTG (for selected clones) for overnight. The over night cultures were spun, and the supernatants were transferred to the fresh tubes. The pellets were used to isolate plasmid with Qiagen plasmid kit.

For β-lactamase activity assay, the chromogenic substrate nitrocefin (Calbiochem) was added to the supernatants to a final concentration of 100 μm, and the increase in absorbency at OD486 nm was monitored by microplater reader (Molecular Devices).

Transient transfections and β-lactamase activity assay. Hela cell lines or 293 cell lines (purchased from ATCC) were transfected with plasmids using lipofectine or lipofectamine (Life Technologies). After 24 hours, the supernatants were transferred to the fresh 96-well plate. The chromogenic substrate nitrocefin (Calbiochem) was added to a final concentration of 100 μm, and the increase in absorbency at OD486 nm was monitored by microplater reader (Molecular Devices).

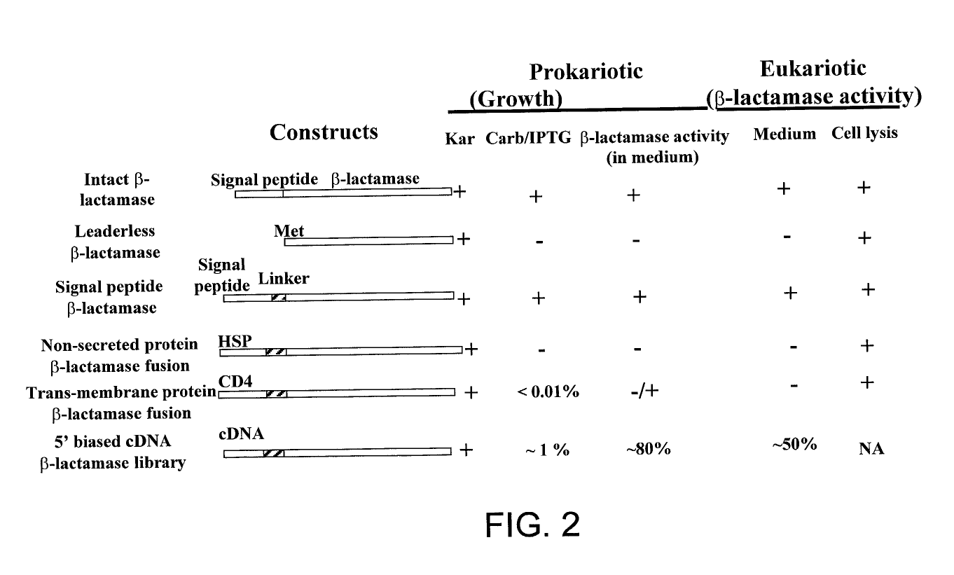

The results of the selection are shown in FIG. 2. The constructs having a nucleic acid encoding a signal peptide inserted 5′ to the leaderless β-lactamase displayed growth in an ampicillin media, whereas the constructs without nucleic acids encoding the signal peptide showed little or no growth. This indicates that use of a vector encoding a cDNA fusion containing a signal peptide has the ability to identify a protein having a signal sequence via secretion of the cDNA-leaderless β-lactamase encoded protein.

### Example 2

### Generation of a pBK-CMV-cDNA-β-Lactamase Library.

Synthesized 5′ biased cDNA was produced as described in U.S. Pat. No. 6,083,727. The generated cDNAs were inserted between EcoRI and NotI sites of the pBK-CD4-β-lactamase vector. Following insertion, these cDNAs were selected as described in Example 1, and the cDNA sequences contained within the selected bacteria were analyzed. Approximately 1% of all transformed bacterial clones were selected. A signal peptide was confirmed in 50-60% of the clones analyzed. Of these, 40% were novel proteins. Validation of the secreted proteins via transfection into mammalian cells indicated that at least half of the selected proteins are secreted in mammalian cells.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. A method of identifying a nucleic acid encoding a signal sequence, the method comprising:
directionally introducing a cDNA into a vector comprising a nucleic acid encoding a leaderless secretable selection protein to produce a fusion nucleic acid insert in said vector, the fusion nucleic acid encoding a fusion protein;
introducing the vector comprising the fusion nucleic acid into a bacterial cell, said introducing allowing for expression of the fusion protein;
exposing the bacterial cell to a selection medium, wherein said selection medium supports growth of bacteria that secrete the fusion protein; and
determining growth of the bacterial cells in said selection medium;
wherein growth of the bacterial cells in said selection medium indicates that the nucleic acid encodes a signal sequence.

2. The method of claim 1, wherein the vector is a dual expression vector.

3. The method of claim 2, wherein the vector comprises a mammalian promoter and a bacterial promoter.

4. A method of identifying a nucleic acid encoding a signal sequence, comprising:
directionally introducing a CDNA into a vector comprising a nucleic acid encoding a leaderless β-lactamase to produce a fusion nucleic acid insert in said vector, the fusion nucleic acid encoding a fusion protein;
introducing the vector comprising the fusion nucleic acid in a bacterial cell, said introducing allowing for expression of the fusion protein;
exposing the bacterial cell to a selection medium; and
determining growth of the bacterial cells in said selection medium, wherein the selection medium supports growth of bacteria that secrete the fusion protein;
wherein growth of the bacterial cells in said selection medium indicates that the nucleic acid encodes a signal sequence.

5. The method of claim 4, wherein the selection medium is a medium comprising β-lactam antibiotic.

6. The method of claim 5, wherein the selection medium comprises ampicillin.

7. The method of claim 4, wherein the vector is a dual expression vector.

8. The method of claim 7, wherein the vector comprises a mammalian promoter and a bacterial promoter.

9. A method of identifying a cDNA encoding a signal sequence, comprising:
directionally introducing a cDNA into a vector, said vector comprising:
a prokaryotic promoter, a eukaryotic promoter, a multiple cloning site, and a nucleic acid encoding a leaderless secretable selection protein, wherein said introducing results in the formation of a fusion nucleic acid;
introducing the vector comprising the fusion nucleic acid into a bacterial cell;
exposing the bacterial cell containing the cDNA to a selection medium;
determining growth of the bacterial cell in said selection medium, wherein growth of the bacterial cells in said selection medium is indicative of a signal sequence in said cDNA;
introducing the vector identified as comprising a signal sequence into eukaryotic cells;
culturing the transfected eukaryotic cells; and
detecting secretion of the cDNA-selection protein fusion in the cell culture;
wherein the vector expresses a fusion protein encoded by the cDNA and the nucleic acid encoding the selection protein.

10. A method of identifying a CDNA encoding a protein having a signal sequence, comprising:
directionally introducing a cDNA into a vector, said vector comprising a prokaryotic promoter, a eukaryotic promoter, a multiple cloning site, and a nucleic acid encoding a leaderless β-lactamase protein, wherein said introducing results in the formation of a cDNA-β-lactamase fusion nucleic acid;
introducing the vector comprising the fusion nucleic acid into a bacterial cell;
exposing the bacterial cell to a selection medium;
determining growth of the bacterial cell in said selection medium, wherein growth of the bacterial cells in said selection medium is indicative of a signal sequence in said cDNA;
introducing the vector identified as comprising a signal sequence into eukaryotic cells;
culturing the transfected eukaryotic cells; and
detecting secretion of the cDNA-selection protein fusion in the cell culture;
wherein the vector expresses a fusion protein encoded by the cDNA and the nucleic acid encoding the selection protein.

11. The method of claim 10, wherein the selection medium is a medium comprising β-lactam antibiotic.

12. The method of claim 11, wherein the selection medium comprises ampicillin.

13. The method of clam 10, wherein the β-lactamase is detected in cell culture using a nitrocefin hydrolysis assay.

14. The method of claim 6, wherein the vector comprises a mammalian promoter and a bacterial promoter.

15. A method of producing a cDNA library enriched for proteins comprising signal sequences, said method comprising:
directionally introducing each of a plurality of cDNAs into a vector, said vector comprising a nucleic acid encoding a leaderless secretable selection protein;
introducing each vector into a bacterial cell to create a library comprising the plurality of cDNAs;
expressing the cDNAs in the bacterial cells; and
selecting bacterial cells containing a cDNA encoding a secreted protein by growth in a selection medium;
wherein the selected bacterial cells are enriched for proteins comprising signal sequences.

16. The method of claim 15, wherein the cDNAs are 5′ biased.

17. The method of claim 15, wherein the bacterial cells are subjected to a second round of selection in a selection medium.

18. A high throughput method of identifying a cDNA which encodes a secreted protein, said method comprising:
directionally introducing each of a plurality of cDNAs individually into a vector comprising a nucleic acid encoding a leaderless secretable selection protein, wherein said introducing results in the formation of a cDNA-β-lactamase fusion nucleic acids in a plurality of vectors;
introducing the plurality of vectors into bacterial cells to create a bacterial cell library; and
selecting bacterial cells containing a cDNA encoding a signal sequence by growth in a selection medium;
wherein growth of the bacterial cells in said medium indicates that the cDNA comprises a signal sequence.

19. The method of claim 18, further comprising the steps of isolating the vector from the selected bacterial cells and identifying the sequence of the cDNA.

20. The method of claim 18, wherein the method further comprises determining the sequence of the CDNA inserts.

21. A method for detecting secretion of a protein comprising a signal sequence, said method comprising the steps:
directionally introducing a cDNA encoding a protein into a vector, said vector comprising a nucleic acid encoding a leaderless secretable selection protein, wherein introducing the cDNA into the cell produces a cDNA-selection protein fusion vector;
introducing the protein fusion vector into a bacterial cell;
exposing the bacterial cells containing the nucleic acid fusion to a selection medium; and
determining growth of the bacterial cells in said selection medium;
wherein growth of the bacterial cells indicate that the cDNA encodes a protein comprising a signal sequence.

22. A method for detecting secretion of a protein comprising a signal sequence, said method comprising the steps:
directionally introducing a cDNA encoding a protein into a vector, said vector comprising a nucleic acid encoding a leaderless β-lactamase protein, wherein introducing the cDNA into the cell produces a cDNA-β-lactamase fusion vector;
introducing the fusion vector into a bacterial cell;
exposing the bacterial cells containing the nucleic acid fusion to a selection medium; and
determining growth of the bacterial cells in said selection medium;
wherein growth of the bacterial cells indicate that the cDNA encodes a protein comprising a signal sequence.

23. A vector for identifying a cDNA insert encoding a protein comprising a signal sequence, said vector comprising a prokaryotic promoter, a eukaryotic promoter, a multiple cloning site, and a nucleic acid encoding a leaderless secretable selection protein.

24. The vector of claim 23, wherein the prokaryotic promoter is a bacterial promoter, and wherein the eukaryotic promoter is a mammalian promoter.

25. The vector of claim 23, wherein the secretable selection protein is β-lactamase.

26. The vector of claim 23, wherein the vector is pBK-CMV-leaderless-β-lactamase.
